(19) United States
(12) Patent Application Publication
(10) Pub. No.: US 2019/0380582 A1
(43) Pub. Date: Dec. 19, 2019

(54) Multi-Modal Body Sensor Monitoring and Recording System Based Secured Health-care Infrastructure

(71) Applicant: Mahesh Mukesh Galgalikar, San Jose, CA (US)

(72) Inventor: Mahesh Mukesh Galgalikar, San Jose, CA (US)

(21) Appl. No.: 15/207,503

(22) Filed: Jul. 12, 2016

(51) Int. Cl. A61B 5/00; A61B 5/08

(52) U.S. Cl. CPC A61B 5/0022; A61B 5/0006; A61B 5/1112; A61B 5/0008; A61B 5/08

(57) ABSTRACT

The present invention provides a system and apparatus for monitoring physiological data, more particularly to a sensor patch and digital framework for generating a virtual representation of an individual's body using IS physiological data. A user of the present invention will be able to actively perform medical and qualitative assessments of their health and generate alerts for significant departures from baseline statistics.

### FIELD OF THE INVENTION

The present invention relates generally to a system and apparatus for monitoring physiological data, more particularly to a sensor patch and digital framework for generating a virtual representation of an individual's body using IS physiological data. A user of the present invention will be able to actively perform medical and qualitative assessments of their health and generate alerts for significant departures from baseline statistics.

### BACKGROUND AND SUMMARY OF THE INVENTION

The proposed medical device system is a multi-modal monitoring and recording system that includes a unique sensor (patch) for continuous long-term recording. The Multi-Modal Body Sensor Monitoring and Recording System uses real time analysis of various body vitals to present a differential diagnosis based on the data recorded. Data is recorded and stored in a buffer memory to capture evidence of a clinically significant event. Occurrence of such an event, which meets pre-programmed parameters, causes the data to be stored in memory, subsequently transmitted to a remote monitoring site, and an immediate response alert to be triggered. The patient, along with others, is made aware of the necessity for follow up. The monitor system includes long term memory, multiple sensors to gather body vitals, and a sophisticated low power communications link for transmitting the data to a central monitoring site where transmitted data is analyzed for significant artifacts.

This invention relates to a wearable multi-modal body sensor and network for continuous health monitoring, selective recording, and transmission of various body vitals such as BPM, minimum of 3 lead ECG, blood pressure, 0 2 saturation, body balance, acoustic response, body temperature, bio impedance, blood pressure, location and the like. Recordings can be selectively timed and centered on the occurrence of clinically significant events. These clinically significant events are detected by the monitoring system, and based on combined acoustic analysis of heart along with other vitals. A continuous log of all body vitals is stored in the system according to various scenarios. Equivalent clinical 12-lead ECG is constructed from the collected data using combined liner transform and Fast Independent component analysis technique.

For many years, wearable medical devices have focused on monitoring and recording electrocardiographic (ECG) information. Cardiac patients have been evaluated with a device known as a “Holter” monitor. The patient wears a series of small sensors which pick up various signals from the heart. These signals are recorded on a paper tape which is sent to a central station for evaluation. A computer may be used to search for irregularities which might have occurred during recording of up to 24 hours or more. These devices have limitations, most notably, a patient may not have been symptomatic during the monitoring period. Thus, the recorded arrhythmias may have little or no significance. Transmission time and subsequent analysis of the data may cause unacceptable delays in critical diagnosis. Overall, the Holter device has many limitations restricting its efficiency.

With advances in technology, medical event monitoring devices have been developed for specific cardiac monitoring. These devices, worn by patients, record ECG information when triggered by the patient or the occurrence of a significant event. The recording usually lasts for one to five minutes and can be transmitted by telephone. The advantage of such a device is the capability for extended monitoring as the device does not have to be continuously operational. A major disadvantage is the availability of limited data to physicians for analysis. They are accustomed to extended monitoring information at hospitals.

Another type of device illustrated in U.S. Pat. No. 4,622,979 (to Katehis et al.) defines an ECG monitoring device which continuously monitors and digitally stores information in memory. When memory becomes full, new data overwrites the old data. Upon occurrence of an event the patient may halt the overwriting of data. Data may then be downloaded via smart phone to a central location for analysis. The device may be programmed to retain a defined time frame of data before and after activation. This device also has a major disadvantage in that it does not have the capability to provide extensive data and/or an extended monitoring period as do Holter type devices. Another major disadvantage is that the device relies on patients to trigger recording. A clinically significant event may occur without the patient being symptomatic (e.g. the patient feels no pain while an event is occurring). Thus no event is recorded.

In an attempt to resolve the above issues, another device U.S. Pat. No. 5,730,143 (to Schwarzberg et al.) was introduced. It was an ECG monitor (Holter type device) and recording device, which includes long-term recording and selective event recording. The selective recording permits real-time evaluation of the incoming data for evaluation of a clinically significant event. The parameters of what constitutes a clinically significant event are adjustable. The device may be remotely programmed in accordance with a physician's orders and based on the patient's medical history. Upon meeting the required parameters the data is stored in an evaluation buffer and the patient is alerted. The patient can manually transfer data to a holding buffer. Data can also be transmitted to a doctor or a central monitoring station. The device also includes long-term data recording like a “Holter’ monitor.

Advancements in the field of telemedicine have brought a revolution in health care monitors. Multiple embedded sensors now monitor various vitals. A device illustrated in U.S. Pat. No. 7,222,054 (to Geva et al.) is directed to personal ambulatory wireless health monitoring for mobile patients. The device contacts a central station to record the patient's physiological data and the patient's location. It can also provide two-way voice communication between the patient and the central station. This device monitors ECG, 02 saturation, blood glucose, body temperature, blood pressure and includes an air flow sensor which measures spirometry. Monitoring may be initiated by the patient with or without a periodic reminder or it may be initiated by programming the device

Most Remote diagnostic products are mainly focused on ECG recording and analysis and over all analytics are single variable based. There are few diagnostic patches as discussed above, which are recording multiple vitals, known as equivital, but are either majorly focused on single variable analytical systems or simply recording the data. None of the above cited prior art record acoustic data. It is possible to come up with a better diagnostic results if multiple variables are taken into account simultaneously. These multiple data points prove very helpful in reducing the possibility of false diagnosis. For example, while observing a tachycardia event using an ECG signal there could be two possible diagnosis. If only an ECG signal is considered for this example, physicians must determine if the patient is undergoing cardiac arrest or if the tachycardia is a normal reaction to hyperactivity of body such as working out or playing a sport. In this scenario having an additional data point can give a definitive result. Co-relative analysis of acoustic and ECG data is unique.

Additionally, the Multi-Modal Body Sensor Monitoring and Recording System evaluates blood pressure based on relative analysis of ECG and photo plethysmography. Comparing different body vitals can lead to various sets of differential diagnosis which are shown in the following flow charts. The devices discussed here derive various differential diagnosis by precisely comparing the captured data with a standard set of clinical databases. Furthermore, the present invention is capable of performing dynamic clinical analysis by normalizing any acquired data points with respect to a patients historical baselines. Device implements various machine learning techniques to understand the base line and general trends in patients body vitals and their relative dependencies. For example, if a patient's normal blood pressure is 130/90, the Multi-Modal Body Sensor Monitoring and Recording System will not generate a clinically significant event alert, despite the fact that 120/80 is considered to be standard. Thus, co-relating different body vitals to determine emotional and physical state of a person could prove to be very helpful in profiling patients, as every patient has their own normal state. This analysis can be very effective in understanding a player's physiology while he is “on the field”. Such player profiling can prevent lot of ‘on field’ injuries of elite athletes. The GPS capability of the Multi-Modal Body Sensor Monitoring and Recording System helps coaches to monitor the activity and vitals of an entire team, and can facilitates improvised strategy planning. The system hardware consist of a monitor device and a disposable sensor patch. Sensor patch can either be a smart fabric sensor patch or traditional adhesive sensor patch.

Another novel thing about the device is, integration of data with cloud infrastructures. This integration makes any acquired healthcare data globally accessible; enabling patients to receive consultations from remotely situated clinical specialists. Cloud integration not only enhances the data analytics capabilities, but also generates a data set of interrelated case studies which facilitate the advancement of medical science. Another major advantage of the present invention is the integration of proprietary cloud services with electronic heath records. This enables doctors to access patient history with ease and get a well maintained and detailed patient profile about what is his normal physical state and how the patient reacts to situations based on the data captured by our device in his natural habitat.

The invention presented herein is directed to overcome the shortcomings of the “Holter” type devices, event type cardiac monitors, and personal ambulatory wireless health monitors.

In accordance with the present disclosure, an efficient and low cost multi-model health monitoring system including real time analysis of various human body vitals is provided. Various embodiments of the disclosed subject matter provide a system to sense various body parameters, continually monitor, selectively record data, transmit various body vitals and provide analysis in real time of that data to provide unique set of differential diagnosis, which are not limited to the ones presented in the flow charts. The data is transmitted via a sophisticated low power communication link through the wireless cellular telephone network. One embodiment of the enclosed subject provides a wearable acoustic patch device with the capability to identify twenty-one different heart abnormalities. This Acoustical Pattern Recognition (APR) system is based on a wearable a sensor acquiring heart sounds and through complex signal processing detecting and differentiating various heart defects. The acquired data is compared with the other body vitals to come up with diagnostic solutions. An alternative embodiment of the disclosed subject matter provides a more comprehensive way of reconstructing 12 lead clinical ECG from just 3 Lead ECG data using complex combination of LT and fast-ICA technique. An alternative embodiment of the disclosed subject matter provides a more comprehensive multi-modal sensor system capable of monitoring and recording data related to body vital functions such as, but not limited to, BPM (heart rate), HRV, ECG, blood pressure, 02 saturation, and body balance and gait, body temperature.

These and other advantages of the disclosed subject matter, as well as additional novel features, will be apparent from the description provided herein. The intent of this summary is not to be a comprehensive description of the claimed subject matter, but rather to provide a short overview of some of the subject matter's functionality. Other system, methods, features and advantages here provided will become apparent to one with skill in the art upon examination of the following figures and detailed description. It is intended that all such additional systems, methods, features and advantages be included within this description, and be within the scope of the accompanying claims.

In accordance with the preferred embodiment of the present invention including a portable adhesive or smart sensor fabric patch including at least one physiological data sensor node operative to gather physiological data of the patient, onboard GPS modality to capture the exact location of the patient or player in the field. Bluetooth or Zigbee or any RF protocol for communication with the central processing hub, digital signal circuitry for processing signals associated with any physiological data form sensor nodes.

Further in accordance with a preferred embodiment of the present invention at least one physiological data sensor node is assembled within the patch. Still further in accordance with a preferred embodiment of the present invention at least one physiological data sensor node is assembled partially external to the patch. Additionally, in accordance with a preferred embodiment of the present invention the external portion of at least one physiological data sensor node is connected to the patch via a connector.

Further in accordance with a preferred embodiment of the present invention the Personal status monitor operates the sensor nodes on polling basis. Further in accordance with a preferred embodiment of the present invention at least one physiological data sensor node operates continuously. Additionally, in accordance with a preferred embodiment of the present invention the multi-modal patch includes memory for storing any of the physiological data. Moreover, in accordance with a preferred embodiment of the present invention the multi-modal patch memory includes standard reference database for comparison with the physiological data recorded by sensor nodes.

Additionally in accordance with a preferred embodiment of the present invention the multi-modal patch the system contact the emergency services, central hub, patient's relative when physiological data appears to be outside the normal parameters.

### BRIEF DESCRIPTION OF THE DRAWINGS

FIG. 1 shows several views, as well as an illustrated schematic of the Multi-Modal Body Sensor Monitoring and Recording System.

FIG.2 depicts the intended method of use for the sensor patch: attached to an individual's body.

FIG.3 is a diagram illustrating the system overview of the present invention.

FIG.4 is a diagram illustrating the system architecture of the present invention.

FIG.5 is a diagram illustrating the system data flow of the present invention.

FIG.6 is a diagram illustrating the system overview of the present invention.

FIG.7 is an illustration of one embodiment of the user interface for the system control program used in the present invention.

FIG. 8 is a diagram illustrating the data processing overview of the present invention.

FIG. 9 is a diagram illustrating the system overview of the present invention.

FIG. 10 is a diagram illustrating the communication protocol used in the present invention.

FIG, 11 is a diagram illustrating the method used to differentiate significant points in the present invention.

FIG. 12 is an illustration of a possible chart containing patient centric data acquired by the present invention.

FIG. 13 is a diagram illustrating a method used for patient assessment in the present invention.

FIG. 14 is a diagram illustrating a method used for patient assessment in the present invention.

FIG. 15 is a diagram illustrating a method used for patient assessment in the present invention.

FIG. 16 is a diagram illustrating a method used for patient assessment in the present invention.

FIG. 17 is a diagram illustrating a method used for patient assessment in the present invention.

FIG. 18 includes several charts illustrating aortic acoustic data collected by the sensor patch of the present invention.

FIG. 19 includes several charts illustrating pulmonic acoustic data collected by the sensor patch of the present invention.

FIG. 20 includes a chart illustrating pulmonic acoustic data collected by the sensor patch of the present invention.

### DETAIL DESCRIPTIONS OF THE INVENTION

All illustrations of the drawings are for the purpose of describing selected versions of the present invention and are not intended to limit the scope of the present invention.

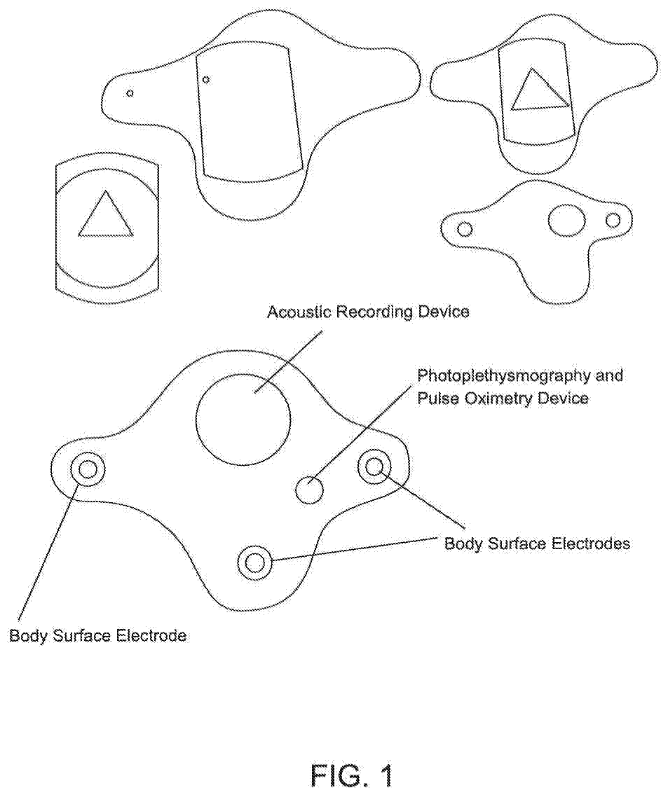

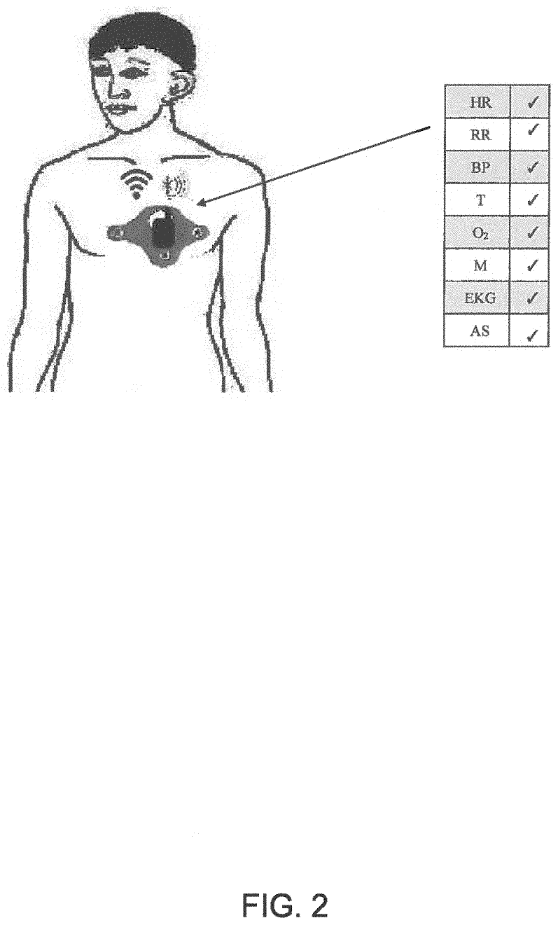

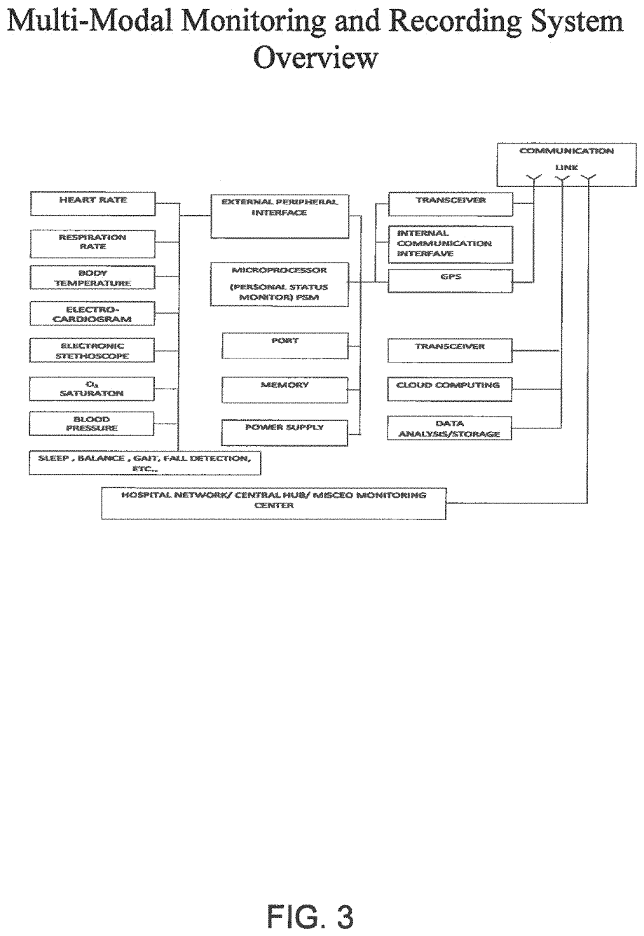

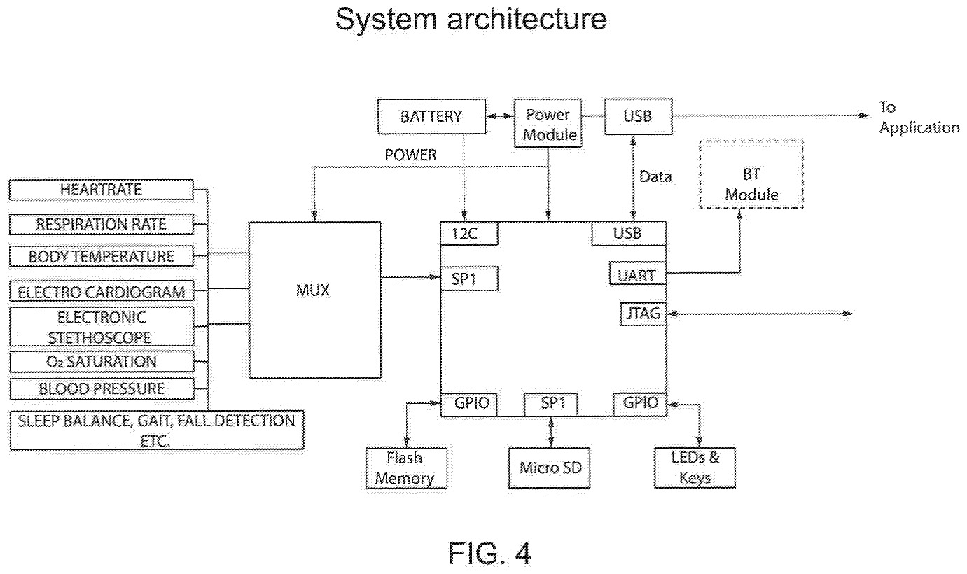

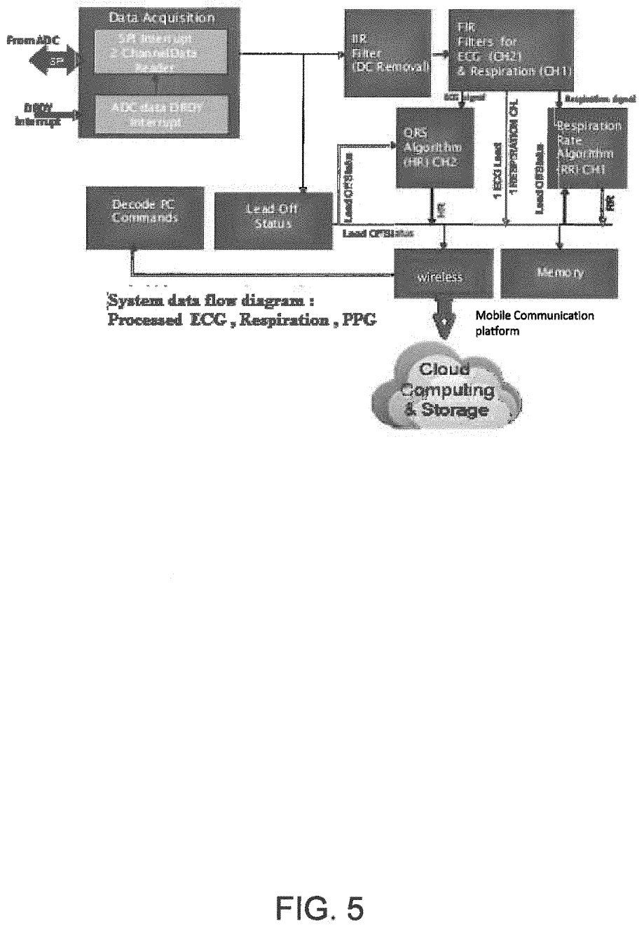

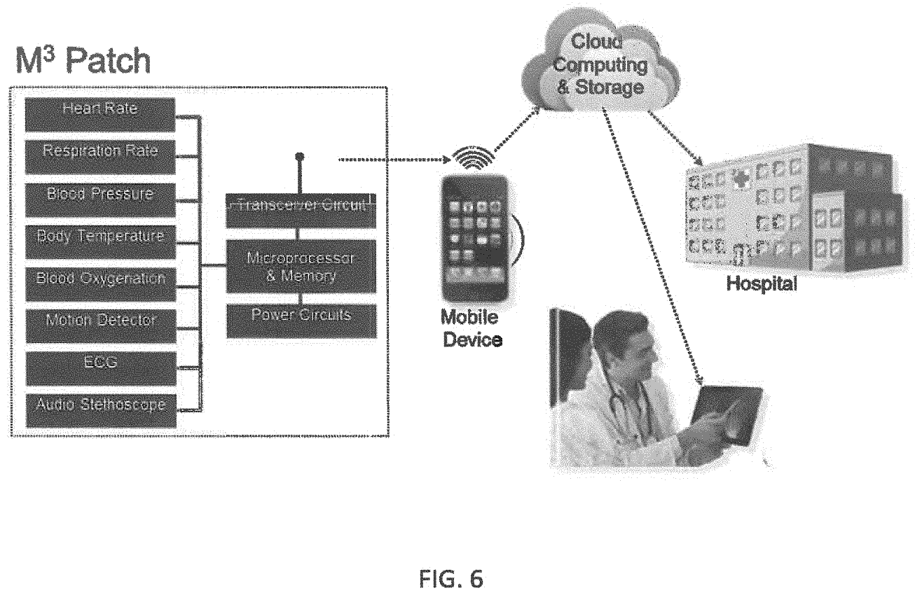

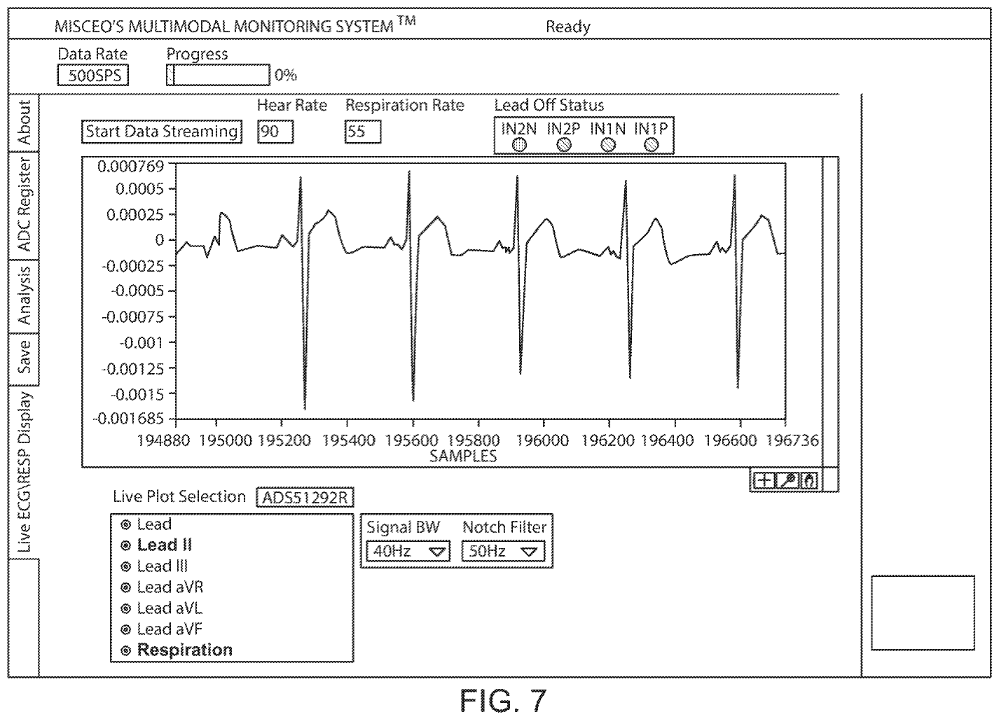

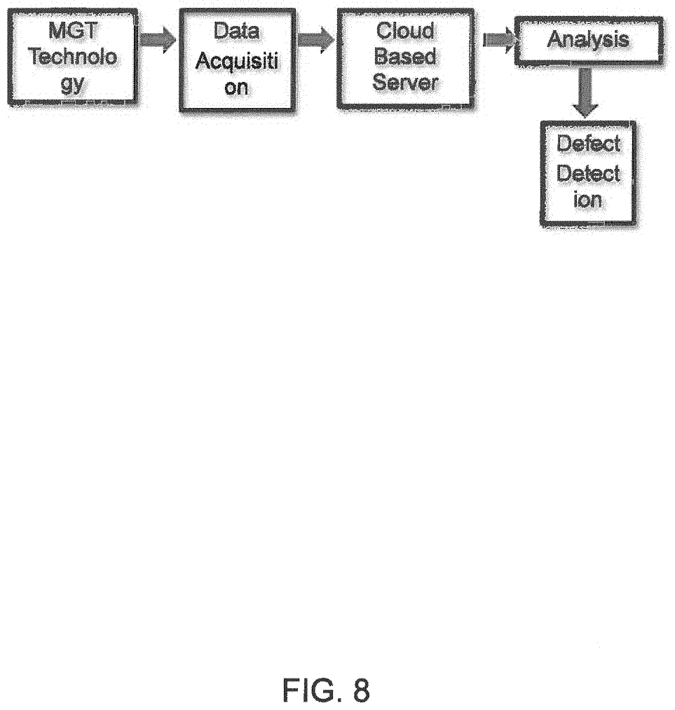

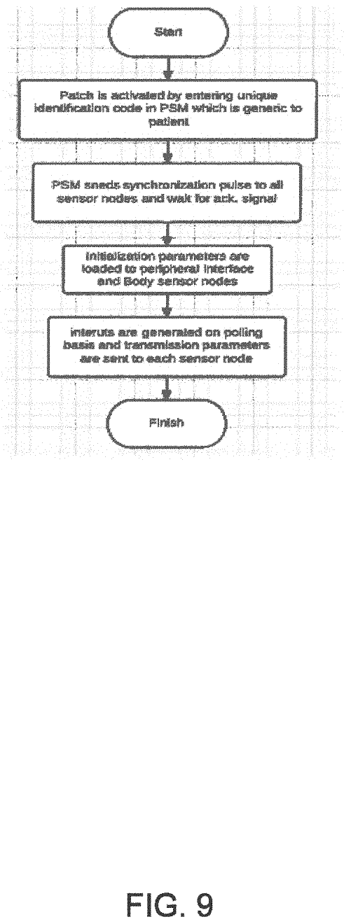

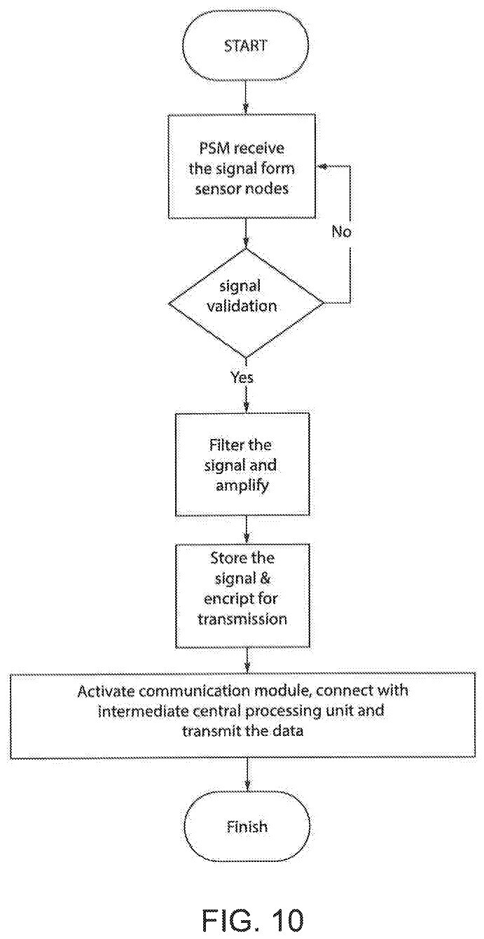

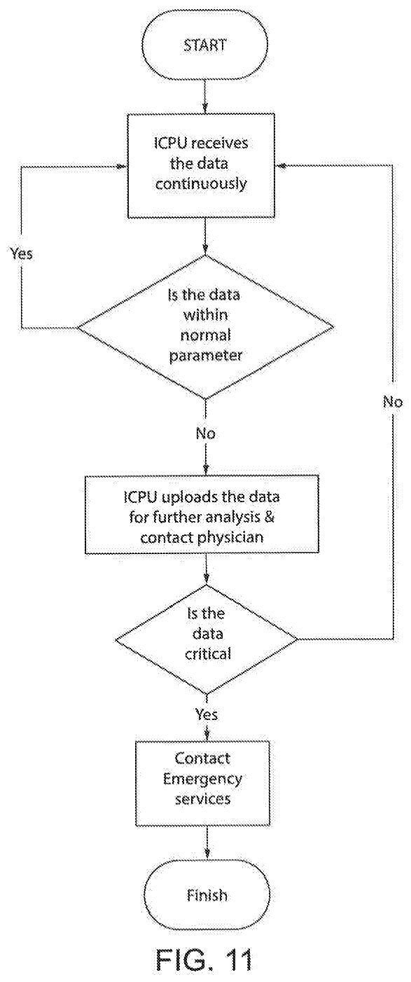

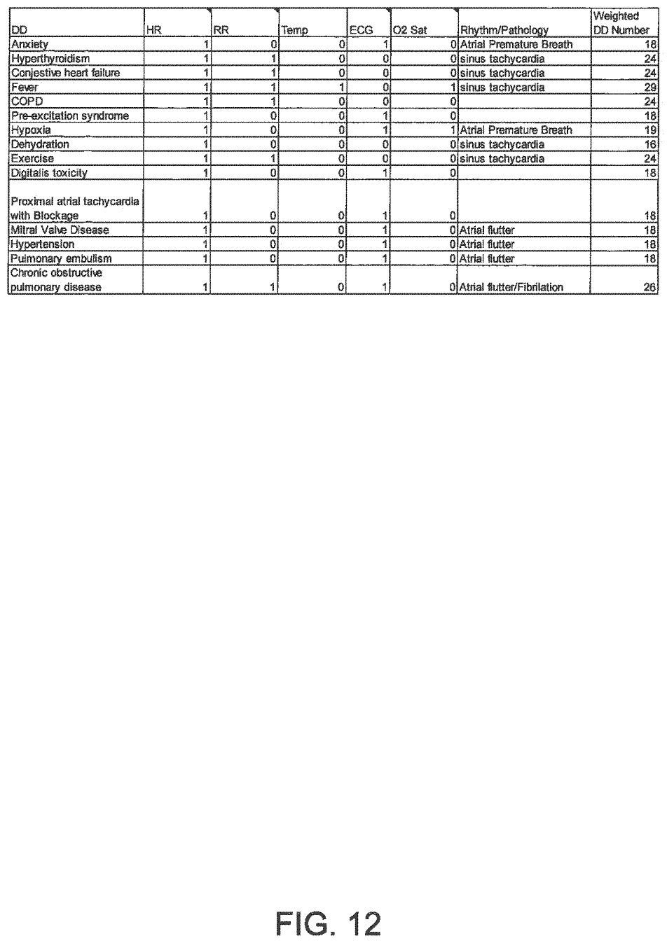

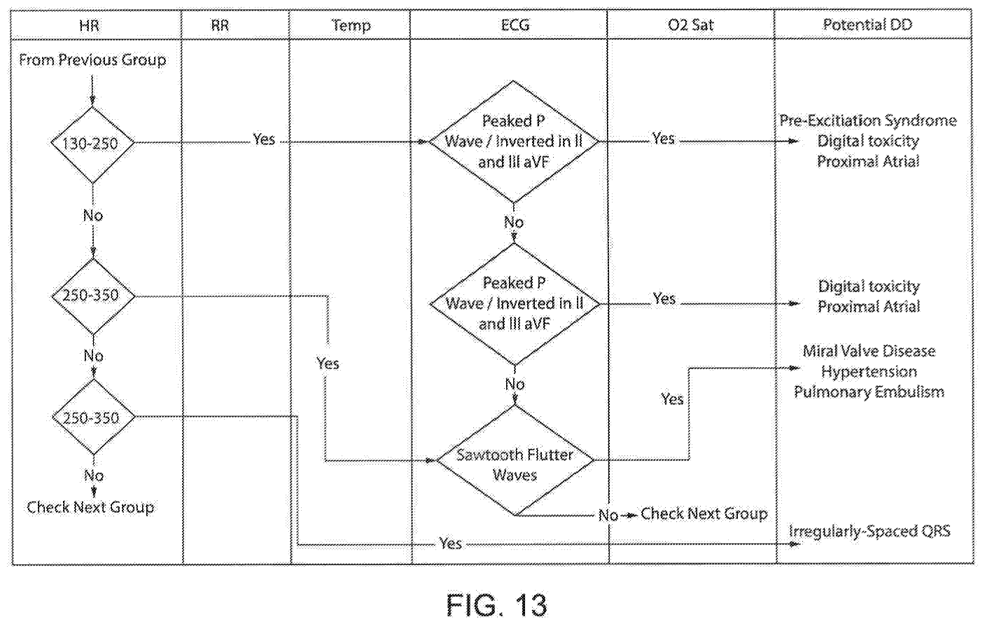

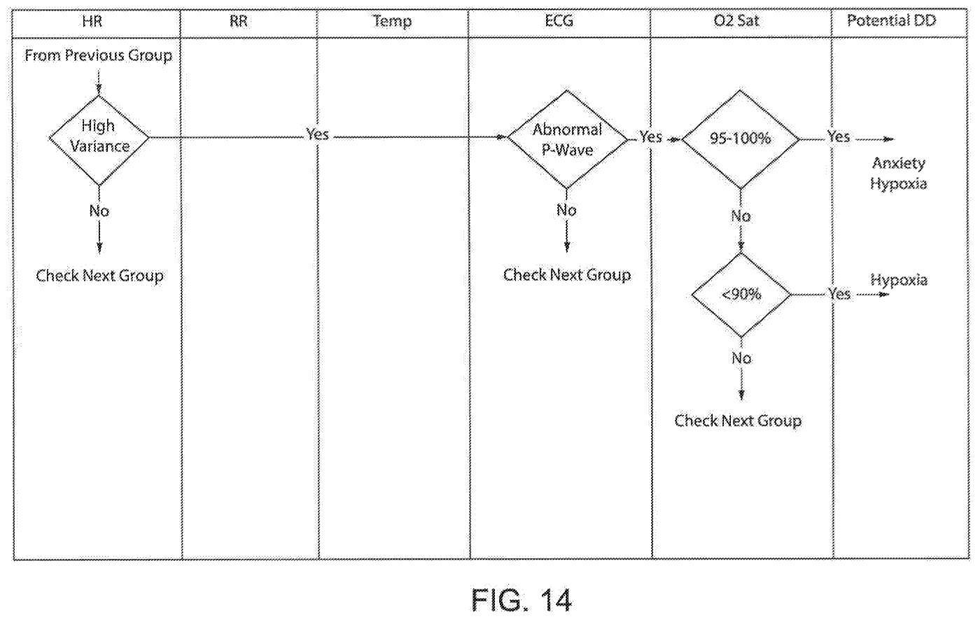

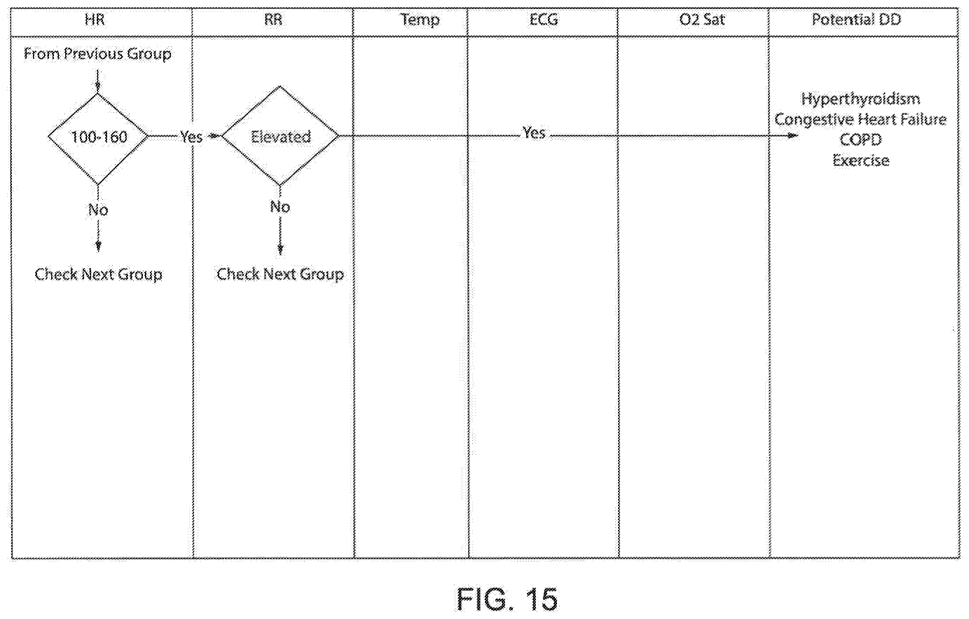

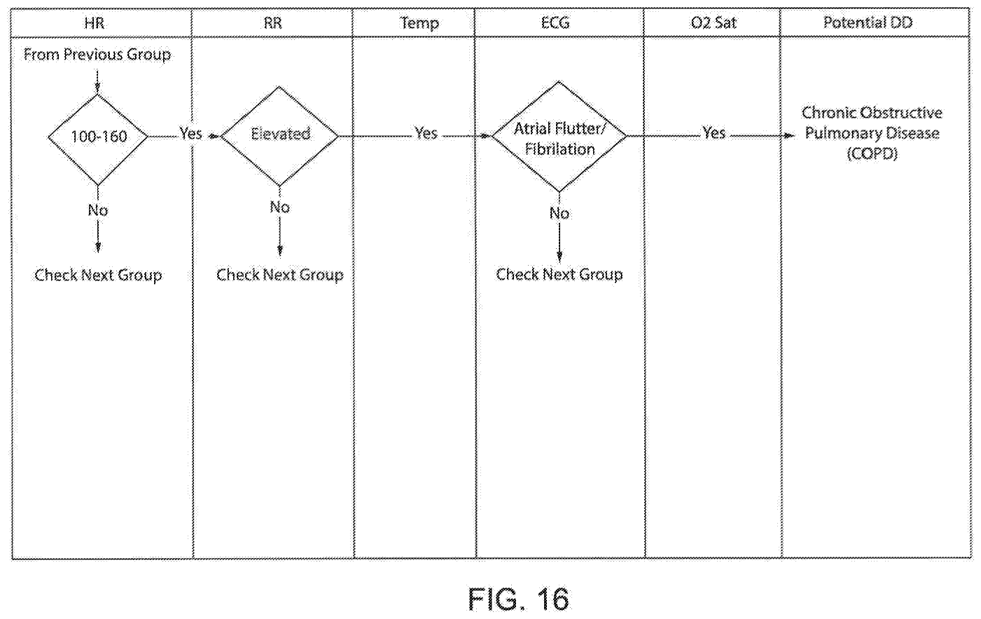

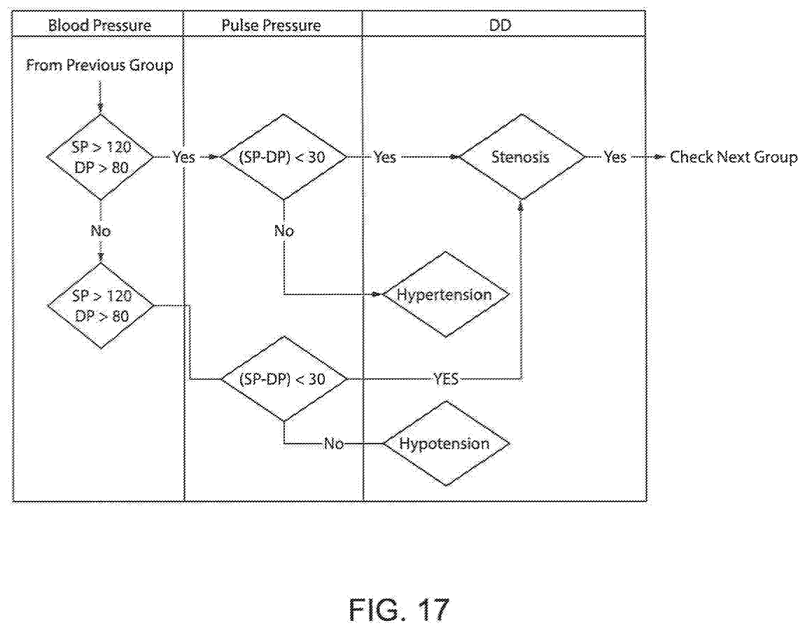

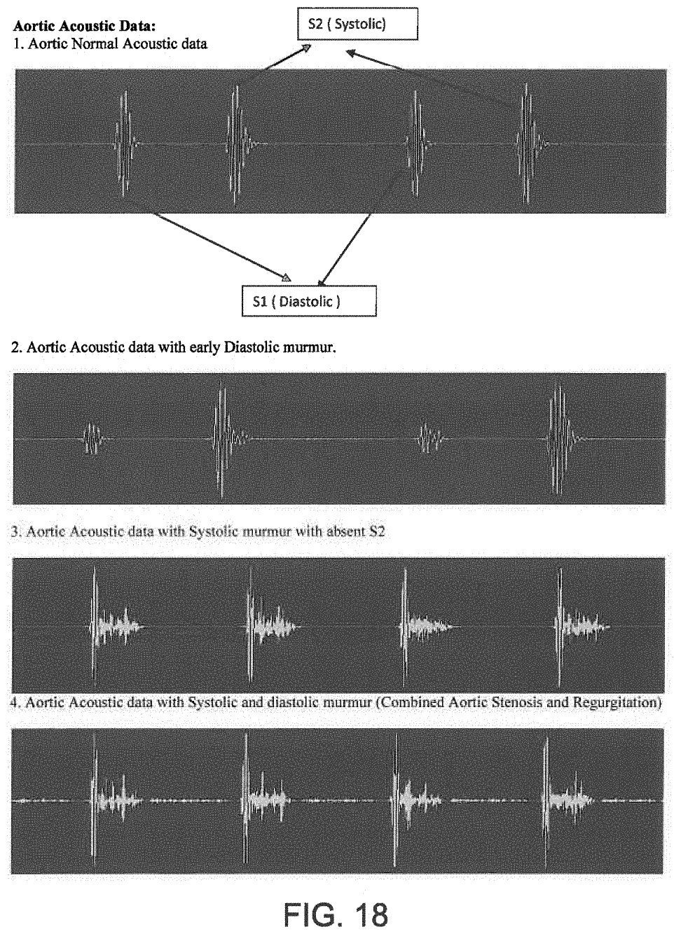

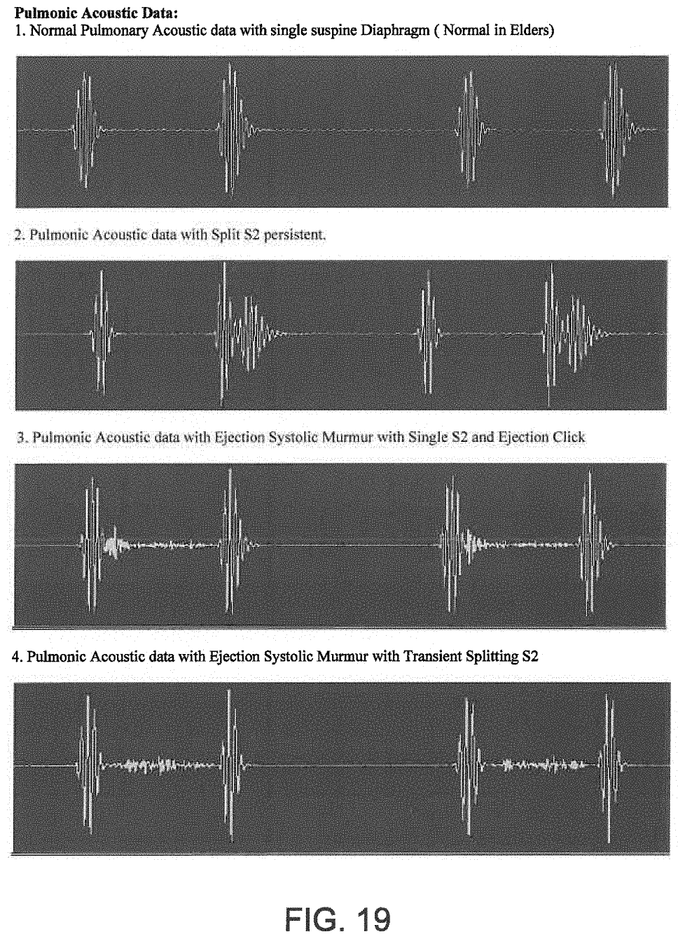

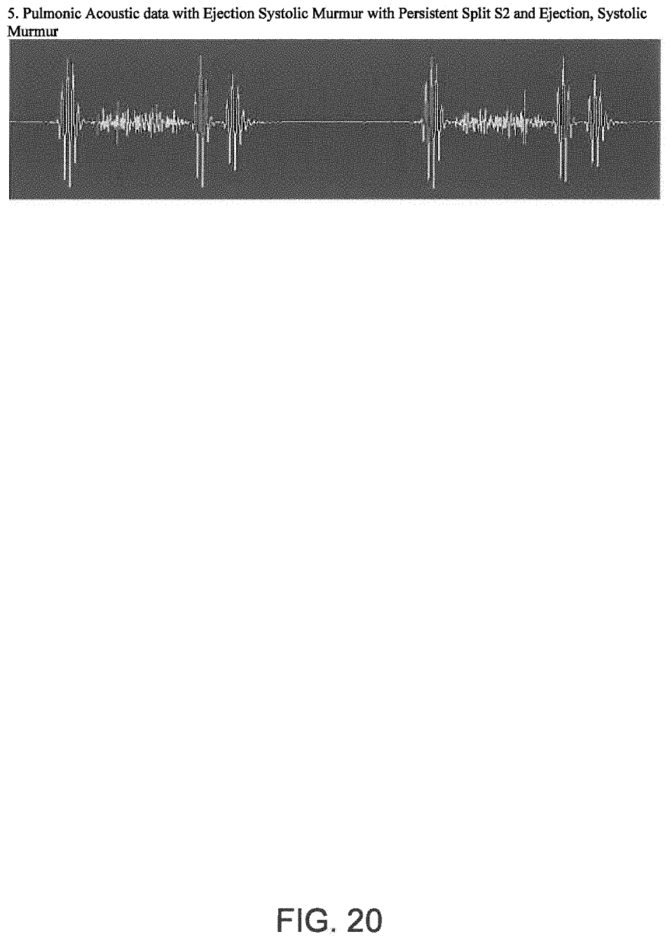

The Following descriptions are in reference to FIG. 1 through FIG. 20. The preferred embodiment of the present invention, the Multi-Modal Body Sensor Monitoring and Recording System, is a system and apparatus which enables remote tracking and multi-component analysis of physiological data. The Multi-Modal Body Sensor Monitoring and Recording System comprises a network connected database, a system control program (SCP), and a personal status monitor (PSM). It is an aim of the present invention to provide a telemedicine device which uses a multi-point sensing device, a multi-platform system control program, and a network connected database to create a platform which enables users to construct virtual models of their overall health. The PSM, when attached to the body of a user, relays said user's biometric data to the database and smart devices running the SCPo The term smart devices is used herein to refer to any electronic device with at least a screen, a means of user input, a network connection, and the capability to effectively run the SCP. In the preferred embodiment of the present invention, the database is a remotely situated data center which is used to process requests for stored data. This datacenter is used to provide cloud computing and storage services for the Multi-Modal Body Sensor Monitoring and Recording System.

The SCP uses the data collected by the PSM's sensor array to construct a virtual model of an individual's quantifiable biological markers. This virtual model is used assess whether changes in a user's physiology should be construed as a clinically significant event. Because the PSM is used to track the biometric data of an individual, the PSM is able to identify clinically significant events which represent significant departures from an individual's baseline vitals. In the preferred embodiment of the present invention, the SCP uses adaptive machine learning algorithms to dynamically alter the criteria for data which signifies a clinically significant event based on changes in a user's environment and activity level. That is, the SCP uses real-time sensor information, as well as an individual's stored biometric data, to make accurate assessments of whether or not auser is in need of medical assistance. In addition to detecting clinically significant events for an individual, the SCP is capable of performing analysis of groups of individuals wearing PSM devices. This functionality enables authorized individuals to track the physiology of teams who are performing group activities.

In a preferred embodiment of the present invention, the system control program is an application which enables users to access physiological data which are used to track quantified measurements of an individual's medical condition. The SCP comprises a profile engine, an assessment engine, a communications engine, and a user interface engine. It is an aim of the present invention to provide an SCP equipped with an applications programming interface (API) suite which enables the program to integrate the functionalities of various electronic health systems into a single platform.

The term engine is used herein to refer to collections of programs which are grouped according to function. Additionally, the term suite is used to denote specialized subgroups of programs within a more generalized programming engine. In the preferred embodiment of the present invention the profile engine is the collection of programs responsible for associating individual users with medical data. Each user of the Multi-Modal Body Sensor Monitoring and Recording System creates a unique profile which is given authorization to access and modify the user's protected health information (PHI). In the Multi-Modal Body Sensor Monitoring and Recording System all physiological assessment data and electronic medical records information are tied to, and cannot be accessed without authorization from, a user's profile. That is, after a user has created a profile for the Multi-Modal Body Sensor Monitoring and Recording System the SCP is granted permissions to access a user's medical information, such as medication history, insurance coverage, demographic information, medical records, and the like. These permissions are used to access the protected medical information provided by different types of electronic medical systems. The profile engine comprises a machine learning suite which dynamically generates a virtual model of a user's physiology. In a preferred embodiment of the present invention, the machine learning suite uses physiological data collected over extended periods of time to actively modify how the SCP interprets real-time sensor output.

In a preferred embodiment of the present invention the assessment engine is tasked with classifying data collected by the sensor array, performing analysis on this data to determine clinically significant events, and tracking user data. The assessment engine comprises a biometrics assessment suite, a correlation suite, and a tracking suite. It is an aim of the present invention to provide an assessment engine which generates quantifiable measures of users ‘physiology while participating in physical activities and medical assessments. That is, the assessment engine generates standardized metrics of how individual's bodies are responding to external stimuli, physical exertion, and medical assessments. These metrics are used to provide personalized representations of how an individual's body is functioning. Furthermore, these personalized metrics are used to assess if a user is experiencing a clinically significant event which should generate an alert. Additionally, the assessment engine uses this data to provide targeted insights of how to modify an individual's lifestyle to achieve desired outcomes.

In a preferred embodiment of the present invention, the biometrics assessment suite performs independent component analysis to reconstruct clinical 12 lead ECG from 3 lead ECG captured by Multi Modal Body Sensor Monitoring and Recording System. This analysis enables the assessment engine to assess an individual's health using multiple points of data. These multiple data points enable the SCP to form relevant judgements as to whether or not an individual's vital signs relate to a clinically significant event. Collected data and any pertinent analysis of said data is stored on the database. Additionally, the biometrics assessment suite is able to integrate data generated by third party devices, and databases into any analysis performed. That is, the biometrics assessment suite used to access, and integrate the clinical data gathered from disparate medical systems.

First an individual selects the desired assessment to be performed using the user interface. This command is transmitted to the communications engine which either locates the desired module on the database, or uses the API suite to acquire the requisite information from a third party system. Next, the biometrics assessment suite data is passed to the formatting suite, which uses the acquired data to generate an interactive user interface. Thus individuals using the SCP are able to have a cohesive experience when performing analysis using the Multi-Modal Body Sensor Monitoring and. Recording System. Information gathered by the assessment engine is automatically uploaded to the database and can be accessed by the Multi-Modal Body Sensor Monitoring and Recording System.

In a preferred embodiment of the present invention the correlation suite is tasked with identifying connections between user profile data and objective measurements of physical health or performance. The correlation suite enables authorized individuals to perform analysis which aggregates the medical data from entire populations. That is, the correlation suite enables authorized individuals to perform statistical analysis on the medical data associated with individual users, as well as the aggregated data of large numbers of users. The tracking suite enables authorized individuals to perform longitudinal studies of users ‘physiology. This functionality enables users to gain insight into how their bodies have changed over time. In the preferred embodiment of the present invention, the results of the analysis performed by the analysis, correlation, and tracking suites is used by the formatting suite to generate charts and informative graphical depictions which are displayed via the user interface. These charts are saved in files which can be opened by third party programs.

In a preferred embodiment of the present invention the communications engine is tasked with coordinating the data transferred between devices running the SCP, third party systems, and the database. The communication engine comprises an API suite, an interoperability suite, and a telemedicine suite. It is an aim of the present invention to provide an API suite which enables users to add additional functionalities to the Multi-Modal Body Sensor Monitoring and Recording System by building software interfaces between the SCP and third party applications. Data which is acquired by the API suite is passed to the formatting suite, where it is reconfigured and then displayed via the user interface. Using the API suite it is possible to integrate the services of systems. Embodiments of the present invention are designed to use the API suite to provide functionalities such as electronic prescribing, searching for health care professionals, accessing insurance information, reviewing medical records and the like. The interoperability suite coordinates the secure exchange of information between the devices running the virtual services program, the database, and third party systems. The telemedicine suite enables users to video conference with healthcare professionals.

In a preferred embodiment of the present invention the user interface engine generates the graphical interface which users interact with. The user interface engine comprises a formatting suite and an input suite. It is an aim of the present invention to provide a user interface engine which interprets and executes user commands. The formatting suite is tasked with reconfiguring the data acquired through the assessment and communications engines, such that this data is used to present the user with a uniform experience. That is, all information processed and presented to a user via the display of a device running the SCP is formatted by the formatting suite. In the preferred embodiment of the present invention, the formatting engine is tasked with generating the graphical interfaces with which users interact. The input suite interprets and executes user commands. User input from physical keys and touchscreen interfaces are interpreted by the input suite. These input commands are then executed by the SCP.

In a preferred embodiment of the present invention the personal status monitor is a device intended to collect information from the Multi Modal Body Sensor Monitoring and Recording, which is relayed to a remotely situated datacenter. The Multi Modal Monitoring Patch comprises a sensor array patch, and a controller device. It is an aim of the present invention to provide a sensor patch which houses the requisite electronic components to accurately measure a user's vital signs. The sensor patch and controller device work in tandem, such that the controller device contains the data processing and communication components which dictate the operation of an attached sensor patch. Furthermore, the controller device detachably connects to the sensor patch, enabling users to dispose of defective sensor patches or controller devices without replacing the entire apparatus. In one embodiment of the present invention, the sensor patch is designed as a disposable mount for the controller device.

In a preferred embodiment of the present invention the sensor patch is a medical device which is intended to maintain an array of sensors in close proximity to the skin of a user. The sensor patch comprises a sensing face, an intermediary circuit, and a mounting face. It is an aim of the present invention to provide a sensor patch which is a flat piece of material with an array of sensors arranged on its first, or sensing, face. The second, or mounting, face of the sensor patch is designed with a receptacle to which the controller device is connected. The array of sensors on the sensing face is maintained in electrical communication with the controller device mount via the intermediary circuit. In the preferred embodiment of the present invention the sensing face comprises a connective fastener and a sensor array. The connective fastener maintains the sensor patch in a desired position adjacent to a user's body. In one embodiment of the present invention the connective fastener is an adhesive material which is placed around the perimeter of the sensor patch and causes the sensor patch to become fixedly attached to an individual. In a separate embodiment, the connective fastener is a strap which extends from the sides of the sensor patch. In this embodiment the strap secures the sensor patch to a patient's body by wrapping around the desired body part and forming a mechanical connection. Embodiments of the sensor patch are integrated into articles of clothing which maintain the PSN in desired positions. In the preferred embodiment of the present invention the sensor array is a collection of sensors which actively monitor the quantifiable biological markers of an individual's body. The sensor array comprises at least a BPM (heart rate) sensor, an ECG sensor, RR (Respiration rate sensor) a blood pressure sensor, photoplethysmography (PPH) sensor, an accelerometer, a body temperature sensor, and an acoustic recording device.

In a preferred embodiment of the present invention the intermediary circuit connects the sensors of the sensor array to the data connection of the controller mounting device. Data and electrical power is relayed between the sensor array and a connected controller device through the intermediary circuit. The mounting face is the face of the sensor patch which is opposite the sensing face and comprises a controller device mount and a data connection. in the preferred embodiment of the present invention the mounting face functions as the connection point by which a controller device is connected to the sensor patch. The controller device mount forms a mechanical connection with the sensor patch attachment mechanism of the controller device. Ernbodiments of the Multi-Modal Body Sensor Monitoring and Recording System are designed with a controller device mount which use fastening mechanisms such as latches, clamps, clips, hooks, buttons and the like. In a separate embodiment the controller device mount is a receptacle into which the controller device is placed and retained. In the preferred embodiment of the present invention, the data connection is an electrical interface which is used to maintain the patch interconnect of the controller device in electrical communication with the intermediary circuit of the sensor patch. A controller device which is inserted into the controller device mount is maintained in a desired position relative to the sensor patch until this connection is disengaged.

In a preferred embodiment of the present invention, the controller device is a module which contains the electrical components required to dictate the functions of the sensor patch, perform preliminary data analysis, and communicate with external devices. The controller device comprises a housing, a removable access panel, a system on a chip (SoC), a wireless radio, a GPS module, a power supply, and a data port. It is an aim of the present invention to provide a controller device which is capable of connecting to and controlling sensor patches designed with various arrangements of biological sensors. That is, the software and firmware of the controller device can be modified to accommodate the processing requirements associated with controlling a wide range of disparate sensors. The housing is a rigid enclosure which encompasses the components of the controller device. The housing comprises a sensor patch attachment mechanism and a controller cavity. The sensor patch attachment mechanism functions as described and serves as a fastener which is used to affix the controller device to the controller device mount of a sensor patch. The sensor patch attachment mechanism comprises a patch interconnect. In the preferred embodiment of the present invention, the patch interconnect is used to deliver electrical power and computational instructions to the sensor array. In one embodiment of the present invention the patch interconnect and data connection are designed using corresponding male and female connectors. In a separate embodiment the patch interconnect and data connection are conductive plates which are maintained in electrical communication by the connection established by the controller device mount and the sensor patch attachment mechanism.

In a preferred embodiment of the present invention, the controller cavity is a compartment within the housing where the electronic components of the controller device are stored. Users are able to access the controller cavity by disengaging the panel fasteners, which maintain the removable access panel in a closed position; sealing the controller cavity. That is, the removable access panel is a piece of material that, when removed, exposes an opening in the walls of the housing. This opening grants users access to the controller cavity within the housing.

In a preferred embodiment of the present invention, the SoC functions as the central processor for the Sensor patch. The SoC comprises a control circuit and onboard memory. It is an aim of the present invention to provide a control circuit that functions as the input output bus through which data is communicated between the SoC, the electronic components of the controller device, and the sensor array of a connected sensor patch. The control circuit is tasked with maintaining the SoC in electrical communication with the patch interconnect, and the data port. The onboard memory functions as the local storage for data acquired by the sensor array, as well as programs and routines which dictate the functions of the PSM. In a separate embodiment of the present invention the onboard memory is augmented by removable storage media.

In a preferred embodiment of the present invention, the controller device is designed with a wireless radio capable of connecting to and communicating over wireless networks. Embodiments of the present invention are manufactured with wireless radios which communicate using standardized wireless protocols such as RFID, ZigBee, Wi-Fi, Bluetooth, GSM, LTE, Wi-Max, NFC, and the like.

In a supplementary embodiment the wireless radio enables the PSM to communicate with a plurality of sensors and smart devices. The data from these connected devices is used to construct a detailed profile of an individual.

In a preferred embodiment of the present invention, a GPS module is integrated into the controller device. This module enables the PSM to record both physiological and positional data. The power supply is housed within the controller cavity and comprises a battery and a power circuit. It is an aim of the present invention to provide a power supply capable of providing the requisite power for both the controller device and the sensor patch to function. In the preferred embodiment of the present invention, the battery is a rechargeable battery which is maintained in electrical communication with the electronic components of the controller device via the power circuit. In a separate embodiment the power supply is equipped with energy harvesting systems, which use renewable sources to generate power for the controller device. The data port is an interconnect which enables users to physically connect the controller device to external systems. Both power and data are transmitted through the data port. That is, the controller device can be physically connected to an external power supply through the data port. Additionally, the data port is used to enable external devices to transfer programs to and access the onboard memory of the controller device. In one embodiment the controller device is equipped with a plurality of physical controls which enable users to manually adjust the function of the PSM.

Although the invention has been explained in relation to its preferred embodiment, it is to be understood that many other possible modifications and variations can be made without departing from the spirit and scope of the invention.

1. An Apparatus for remotely monitoring and assessing the status of a human subject, the apparatus comprising:
a pre-programmed microprocessor in communication with at least two sensors, at least one acoustic sensor, a monitor capable of transmitting all the data collected by the sensors to the remote central unit;
a remote central unit capable of communicating with cloud server via wireless, will, cellular network;
energy harvesters capable to harvesting energy for bio-potential and heat generated by body;
a tracking system that tracks the location of patient;
a power supply connected to microprocessor, energy harvesting unit;
a portable transceiver system comprising: Bluetooth transceiver, will transceiver; GSM transceiver;
a remote central unit capable of analyzing the data and sending all the data to remote cloud server;
software that executes the total operation of the system, analyses the data and transmits with minimal delay introduced due to transmission lag and processing lag;
a buffer memory to store the date before its transmitted to remote central unit;
a flash memory capable of storing selective patient data; and
a detachable monitor which connects to disposable sensor patch.

2. The monitoring device in claim 1, wherein the sensor path is capable of detecting acoustic physiological data, respiratory, opto-electric , physiological and/or other information relating to one or more of the following:
a. Acoustic view of the heart of the person;
b. Respiratory response of a person
c. Electrical activity of cardiac muscles;
d. The blood oxygen level of the person;
e. Body surface impedance of a person
f. Body surface temperature of the person;
g. Gait information of a person;
h. Sleep analytic data of a person
i. Body mineral content of a person;
j. Blood pressure of a person
k. Reconstructed 12 Lead ECG of the person.

3. The system in claim 1, comparatively analyses at least two physiological signal to come up with diagnostic solution.

4. A monitoring device as claimed in claim 1, where in the thresholds are pre-configured and is capable of being overridden or reduced in intensity based on to patient's prior medical.

5. A monitoring device as claimed in claim 1 , capable of understanding the unique pattern of various body vitals of the patents and capable of interpreting various inter dependencies within the other body parameters to come up with diagnostic solution.

6. The monitoring system claimed in claim 1, wherein the plurality of electrodes are integrated in the disposable patch or smart sensor fabric vest.

7. The monitoring system claimed in claim 1 is preprogrammed to perform amplification and filtering of various signals acquired by the peripherals.

8. The system of claim 1 wherein the amplification and filtering capabilities comprise of continuous time adaptive filtering circuitry.

9. The monitoring system claimed in claim 1 comprises an impedance plethysmography circuitry capable of performing respiration analysis of the user.

10. The monitoring system of claim 1 wherein the infrastructure comprises of a local network formed of:
a. Personal Status Monitor (PSM)
b. Local server which comprises of a web server
c. Local advance database
d. Secure and redundant Cloud storage.

11. The monitoring system infrastructure as mentioned in claim 10 comprises of a Personal Status Monitor (PSM) which is a device capable of collecting data packets from the sensor patch, wherein the data received by the PSM from the sensor patch comprises of ECG, Accelerometer, Impedance Sensors, Gyroscope, RTC, Analog temperature sensor information, and wherein The packet is base 64 encoded and sent as a JSON response from the PSM.

12. The infrastructure as mentioned in claim 10 comprises of a local server which is a web server designed to handle JSON response including Reverse encoding for the base 64 encoded payload.

13. The system mentioned in claim 10, where in consists of a local advance database that is responsible for temporary data caching for local access, wherein The data stored into the database is made secure using AES encryption.

14. The system mentioned in claim 10, where in data from the local server is pushed onto the cloud into a secure database bucket, wherein Security of the data transfer is ensured using Server Side Encryption in conjunction with secured access control listing.

15. An Apparatus for remotely monitoring and assessing the status of a human subject, the apparatus comprising ef:
a. A pre-programmed microprocessor in communication with at least two sensors, at least one acoustic sensor, monitor capable of transmitting all the data collected by the sensors to the remote central unit.
b. Remote central unit capable of communicating with cloud server via wireless, wifi, cellular network.
c. Energy harvesters capable to harvesting energy for bio-potential and heat generated by body,
d. A tracking system that tracks the location of patient,
e. Power supply connected to microprocessor, energy harvesting unit.
f. A portable transceiver system comprising; Bluetooth transceiver, wifi transceiver; GSM transceiver.
g. A remote central unit capable of analyzing the data and sending all the data to remote cloud server.
h. Software that executes the total operation of the system, analyses the data and transmits with minimal delay introduced due to transmission lag and processing lag.
i. A buffer memory to store the date before its transmitted to remote central unit.
j. A flash memory capable of storing selective patient data.
k. A detachable monitor which connects to disposable sensor patch.
wherein the personal status monitor is capable of receiving all the information from sensor nodes,
1. a cloud server comprising of virtualization tool along with a differential diagnostics engine, an ECG reconstruction engine consisting of algorithms that perform and evaluate the cardiac conditioning of the user based on interdependencies between various body vital parameters, to tabulate various differential diagnosis , subjects stress level, anxiety level, Endurance, fatigue and adaptive performance indices.

16. The apparatus of claim 15 wherein the processing engine incorporates various machine learning techniques to understand the base line of the subject and tabulates the differential diagnostics.
